(19) United States
(12) Patent Application Publication
(10) Pub. No.: US 2025/0325487 A1
(43) Pub. Date: Oct. 23, 2025

(54) NEW COATING SYSTEM (I)

(71) Applicant: DSM IP Assets B.V., Heerlen (NL)

(72) Inventors: Jimmy DOUCOURE, Kaiseraugst (CH); Elger FUNDA, Kaiseraugst (CH); Olivia Brigitte VIDONI, Kaiseraugst (CH); Alexandru ZABARA, Kaiseraugst (CH)

(21) Appl. No.: 18/719,751

(22) PCT Filed: Dec. 15, 2022
(86) PCT No.: PCT/EP2022/086013

§ 371 Date: Jun. 13, 2024

(30) Foreign Application Priority Data
Dec. 17, 2021 (EP) 21215553.5

(51) Int. Cl. A61K 9/28; A23P 10/35; A23P 20/10

(52) U.S. Cl. CPC A61K 9/288; A23P 10/35; A23P 20/11

(57) ABSTRACT

The present invention relates to a new coating system useful for coating particles, which comprise an active ingredient. Such coated particles, when consumed, do show a controlled release in the human or animal body.

### CROSS-REFERENCE TO RELATED APPLICATIONS

This application is the U.S. national phase of International Application No. PCT/EP2022/086013 filed Dec. 15, 2022, which designated the U.S. and claims priority to EP 21215553.5 filed Dec. 17, 2021, the entire contents of each of which are hereby incorporated by reference.

### FIELD

The present invention relates to a new coating system useful for coating particles, which comprise an active ingredient.

Such coated particles, when consumed, do show a controlled release in the human or animal body.

### BACKGROUND AND SUMMARY

Controlled release is a very important property for particles (such as i.e. tablet, granules etc), in that the active ingredient, which is in the particle, can be delivered to the right place in the human or animal gastrointestinal tract, where the active ingredient is then released.

Such active ingredients can be any commonly used ingredients, such as i.e. a pharmaceutical compound (a drug), a vitamin, a mineral, probiotic, enzyme, eubiotic, plant extract and nutraceutical.

The goal was to find a coating layer that allows the release of the active ingredient inside the particle after passing the stomach in the small intestine.

Surprisingly, it was found that when using a coating system to coat a particle, wherein the coating system is an emulsion comprising an
(i) aqueous solution comprising
   a. water, and
   b. at least one gum having emulsifying properties (preferably gum acacia), and an
(ii) oily phase comprising
   a. at least one enteric coating material (preferably at least one wax; more preferably Candelilla wax), and
   b. at least one oil, which is solid at 20° C. (more preferred hydrogenated oil, even more preferred fully hydrogenated oil, most preferably fully hydrogenated rapeseed oil (canola oil)),
      then the so coated particle shows a delayed release so that the release takes place where it is desired.

### BRIEF DESCRIPTION OF THE DRAWINGS

FIG. 1 is a graph showing the results of the release test from Example 1 (without enzymes);

FIG. 2 is a graph showing the results of the release test from Example 1 (with enzymes; 20% coating);

FIG. 3 is a graph showing the results of the release test from Example 1 (with enzymes; 22% coating);

FIG. 4 is a graph showing the results of the release test from Example 1 (with enzymes; 23% coating);

FIG. 5 is a graph showing the results of the release test from Example 2 (without enzymes; 23.2% coating); and

FIG. 6 is a graph showing the results of the release test from Example 2 (with enzymes; 22% coating)

### DETAILED DESCRIPTION

The present invention relates to a coating system (CS), which is an emulsion comprising an
(i) aqueous solution comprising
   a. water, and
   b. at the least one gum having emulsifying properties, and an
(ii) oily phase comprising
   a. at least one wax, and
   b. at least one oil, which is solid at 20° C.

The present invention also relates to a coating system (CS′), which is an emulsion comprising an
(i) aqueous solution consisting of
   a. water, and
   b. at the least one gum having emulsifying properties, and an
(ii) oily phase comprising
   a. at least one wax, and
   b. at least one oil, which is solid at 20° C.

The present invention also relates to a coating system (CS″), which is an emulsion comprising an
(i) aqueous solution comprising
   a. water, and
   b. at the least one gum having emulsifying properties, and an
(ii) oily phase consisting of
   a. at least one wax, and
   b. at least one oil, which is solid at 20° C.

The present invention also relates to a coating system (CS′″), which is an emulsion comprising an
(i) aqueous solution consisting of
   a. water, and
   b. at the least one gum having emulsifying properties, and an
(ii) oily phase consisting of
   a. at least one wax, and
   b. at least one oil, which is solid at 20° C.

The present invention also relates to a coating system (CS1), which is an emulsion consisting of an
(i) aqueous solution comprising
   a. water, and
   b. at least one gum having emulsifying properties, and an
(ii) oily phase comprising
   a. at least one wax, and
   b. at least one oil, which is solid at 20° C.

The present invention also relates to a coating system (CS1′), which is an emulsion consisting of an
(i) aqueous solution consisting of
   a. water, and
   b. at least one gum having emulsifying properties, and an
(ii) oily phase comprising
   a. at least one wax, and
   b. at least one oil, which is solid at 20° C.

The present invention also relates to a coating system (CS1″), which is an emulsion consisting of an
(i) aqueous solution comprising
   a. water, and
   b. at least one gum having emulsifying properties, and an
(ii) oily phase consisting of
   a. at least one wax, and
   b. at least one oil, which is solid at 20° C.

The present invention also relates to a coating system (CS1′″), which is an emulsion consisting of an
(i) aqueous solution consisting of
   a. water, and
   b. at least one gum having emulsifying properties, and an
(ii) oily phase consisting of
   a. at least one wax, and
   b. at least one oil, which is solid at 20° C.

Usually, the emulsion comprises (or consists of) of 45-80 weight-% (wt-%), based on the total weight of the emulsion, of the aqueous (water) phase and 20-55 wt-%, based on the total weight of the emulsion, of the oily phase.

Usually and preferably, the emulsion comprises (or consists of) of 50-80 weight-% (wt-%), based on the total weight of the emulsion, of the aqueous (water) phase and 20-50 wt-%, based on the total weight of the emulsion, of the oily phase.

The present invention also relates to a coating system (CS2), which is the coating system (CS), (CS′), (CS″) or (CS′″), wherein the emulsion comprises 45-80 wt-%, based on the total weight of the emulsion, of the aqueous (water) phase and 20-55 wt-%, based on the total weight of the emulsion, of the oily phase.

The present invention also relates to a coating system (CS2′), which is the coating system (CS), (CS′), (CS″) or (CS′″), wherein the emulsion comprises 50-80 wt-%, based on the total weight of the emulsion, of the aqueous (water) phase and 20-50 wt-%, based on the total weight of the emulsion, of the oily phase.

The present invention also relates to a coating system (CS3), which is the coating system (CS), (CS′), (CS″) or (CS′″), wherein the emulsion consists of 50-80 wt-%, based on the total weight of the aqueous phase, of water and 20-50 wt-%, based on the total weight of the aqueous phase.

The present invention also relates to a coating system (CS3′), which is the coating system (CS), (CS′), (CS″) or (CS′″), wherein the emulsion consists of 45-80 wt-%, based on the total weight of the aqueous phase, of water and 20-55 wt-%, based on the total weight of the aqueous phase.

All percentages always add up to 100.

In another preferred embodiment of the present invention, the aqueous phase of the comprises
65-90 wt-%, based on the total weight of the aqueous phase, of water, and
0.5-10 wt-%, based on the total weight of the aqueous phase, of at least one gum having emulsifying properties, and
up to 15 wt-%, based on the total weight of the aqueous phase, of at least one further auxiliary agent.

In another preferred embodiment of the present invention, the aqueous phase of the emulsion consists of
75-99 wt-%, based on the total weight of the aqueous phase, of water, and
1-25 wt-%, based on the total weight of the aqueous phase, of at least one gum having emulsifying properties, and
up to 15 wt-%, based on the total weight of the aqueous phase, of at least one further auxiliary agent.

In another preferred embodiment of the present invention, the aqueous phase of the emulsion consists of
75-99 wt-%, based on the total weight of the aqueous phase, of water, and
1-25 wt-%, based on the total weight of the aqueous phase, of at least one gum having emulsifying properties.

In another preferred embodiment of the present invention, the aqueous phase of the emulsion comprises
80-99 wt-%, based on the total weight of the aqueous phase, of water, and
1-20 wt-%, based on the total weight of the aqueous phase, of at least one gum having emulsifying properties, and
up to 15 wt-%, based on the total weight of the aqueous phase, of at least one further auxiliary agent.

In another preferred embodiment of the present invention, the aqueous phase of the emulsion consists of
80-99 wt-%, based on the total weight of the aqueous phase, of water, and
1-20 wt-%, based on the total weight of the aqueous phase, of at least one gum having emulsifying properties, and
up to 15 wt-%, based on the total weight of the aqueous phase, of at least one further auxiliary agent.

In another preferred embodiment of the present invention, the aqueous phase of the emulsion consists of
80-99 wt-%, based on the total weight of the aqueous phase, of water, and
1-20 wt-%, based on the total weight of the aqueous phase, of at least one gum having emulsifying properties.

The auxiliary agents used in the emulsions according to the present invention are commonly known compounds. Suitable auxiliary agents are i.e. colors, antifoaming agents, flavors and antioxidants.

Therefore, the present invention also relates to a coating system (CS4), which is the coating system (CS), (CS′), (CS″), (CS′″), (CS1), (CS1′), (CS1″), (CS1′″), (CS2), (CS2′), (CS3) or (CS3′), wherein the aqueous phase of the emulsion comprises
65-90 wt-%, based on the total weight of the aqueous phase, of water, and
0.5-10 wt-%, based on the total weight of the aqueous phase, of at least one gum having emulsifying properties,
and
up to 15 wt-%, based on the total weight of the aqueous phase, of at least one further auxiliary agent.

The present invention also relates to a coating system (CS4′), which is the coating system (CS), (CS′), (CS″), (CS′″), (CS1), (CS1′), (CS1″), (CS1′″), (CS2), (CS2′), (CS3) or (CS3′), wherein the emulsion consists of
75-99 wt-%, based on the total weight of the aqueous phase, of water, and
1-25 wt-%, based on the total weight of the aqueous phase, of at least one gum having emulsifying properties, and
up to 15 wt-%, based on the total weight of the aqueous phase, of at least one further auxiliary agent.

The present invention also relates to a coating system (CS4″), which is the coating system (CS), (CS′), (CS″), (CS′″), (CS1), (CS1′), (CS1″), (CS1′″), (CS2), (CS2′), (CS3) or (CS3′), wherein the aqueous phase of the emulsion consists of
75-99 wt-%, based on the total weight of the aqueous phase, of water, and
1-25 wt-%, based on the total weight of the aqueous phase, of at least one gum having emulsifying properties.

The present invention also relates to a coating system (CS4′″), which is the coating system (CS), (CS′), (CS″), (CS′″), (CS1), (CS1′), (CS1″), (CS1′″), (CS2), (CS2′), (CS3) or (CS3′), wherein the aqueous phase of the emulsion comprises
80-99 wt-%, based on the total weight of the aqueous phase, of water, and
1-20 wt-%, based on the total weight of the aqueous phase, of at least one gum having emulsifying properties, and
up to 15 wt-%, based on the total weight of the aqueous phase, of at least one further auxiliary agent.

The present invention also relates to a coating system (CS4″″), which is the coating system (CS), (CS′), (CS″), (CS′″), (CS1), (CS1′), (CS1″), (CS1′″), (CS2), (CS2′), (CS3) or (CS3′), wherein the aqueous phase of the emulsion consists of
80-99 wt-%, based on the total weight of the aqueous phase, of water, and
1-20 wt-%, based on the total weight of the aqueous phase, of at least one gum having emulsifying properties, and
up to 15 wt-%, based on the total weight of the aqueous phase, of at least one further auxiliary agent.

The present invention also relates to a coating system (CS4′″″), which is the coating system (CS), (CS′), (CS″), (CS′″), (CS1), (CS1′), (CS1″), (CS1′″), (CS2), (CS2′), (CS3) or (CS3′), wherein the aqueous phase of the emulsion consists of
80-99 wt-%, based on the total weight of the aqueous phase, of water, and
1-20 wt-%, based on the total weight of the aqueous phase, of at least one gum having emulsifying properties.

The present invention also relates to a coating system (CS5), which is the coating system (CS4), (CS4′), (CS4′″) or (CS4″″), wherein the auxiliary agent is chosen from the group consisting of colors, antifoaming agents, flavors and antioxidants.

In another preferred embodiment of the present invention, the oily phase of the emulsion comprises
30-70 wt-%, based on the total weight of the oily phase, of at least one enteric coating material, and
30-70 wt-%, based on the total weight of the oily phase, of at least one oil, which is solid at 20° C., and
up to 15 wt-%, based on the total weight of the oily phase, of at least one further auxiliary agent.

In another preferred embodiment of the present invention, the oily phase of the emulsion consists of
30-70 wt-%, based on the total weight of the oily phase, of at least one enteric coating material, and
30-70 wt-%, based on the total weight of the oily phase, of at least one oil, which is solid at 20° C., and
up to 15 wt-%, based on the total weight of the oily phase, of at least one further auxiliary agent.

In another preferred embodiment of the present invention, the oily phase of the emulsion consists of
30-70 wt-%, based on the total weight of the oily phase, of at least one enteric coating material, and
30-70 wt-%, based on the total weight of the oily phase, of at least one oil, which is solid at 20° C.

In another preferred embodiment of the present invention, the oily phase of the emulsion comprises
40-60 wt-%, based on the total weight of the oily phase, of at least one enteric coating material, and
40-60 wt-%, based on the total weight of the oily phase, of at least one oil, which is solid at 20° C., and
up to 15 wt-%, based on the total weight of the oily phase, of at least one further auxiliary agent.

In another preferred embodiment of the present invention, the oily phase of the emulsion comprises
40-60 wt-%, based on the total weight of the oily phase, of at least one enteric coating material, and
40-60 wt-%, based on the total weight of the oily phase, of at least one oil, which is solid at 20° C.

In another preferred embodiment of the present invention, the oily phase of the emulsion consists of
40-60 wt-%, based on the total weight of the oily phase, of at least one enteric coating material, and
40-60 wt-%, based on the total weight of the oily phase, of at least one oil, which is solid at 20° C., and up to 15 wt-%, based on the total weight of the oily phase, of at least one further auxiliary agent.

In another preferred embodiment of the present invention, the oily phase of the emulsion consists of
40-60 wt-%, based on the total weight of the oily phase, of at least one enteric coating material, and
40-60 wt-%, based on the total weight of the oily phase, of at least one oil, which is solid at 20° C.

The present invention also relates to a coating system (CS6), which is the coating system (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″) or (CS5), wherein the oily phase of the emulsion comprises
30-70 wt-%, based on the total weight of the oily phase, of at least one enteric coating material, and
30-70 wt-%, based on the total weight of the oily phase, of at least one oil, which is solid at 20° C., and
up to 15 wt-%, based on the total weight of the oily phase, of at least one further auxiliary agent.

The present invention also relates to a coating system (CS6′), which is the coating system (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″) or (CS5), wherein the oily phase of the emulsion consists of
30-70 wt-%, based on the total weight of the oily phase, of at least one enteric coating material, and
30-70 wt-%, based on the total weight of the oily phase, of at least one oil, which is solid at 20° C., and
up to 15 wt-%, based on the total weight of the oily phase, of at least one further auxiliary agent.

The present invention also relates to a coating system (CS6″), which is the coating system (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″) or (CS5), wherein the oily phase of the emulsion consists of
30-70 wt-%, based on the total weight of the oily phase, of at least one enteric coating material, and
30-70 wt-%, based on the total weight of the oily phase, of at least one oil, which is solid at 20° C.

The present invention also relates to a coating system (CS6′″), which is the coating system (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″) or (CS5), wherein the oily phase of the emulsion comprises
40-60 wt-%, based on the total weight of the oily phase, of at least one enteric coating material, and
40-60 wt-%, based on the total weight of the oily phase, of at least one oil, which is solid at 20° C., and
up to 15 wt-%, based on the total weight of the oily phase, of at least one further auxiliary agent.

The present invention also relates to a coating system (CS6″″), which is the coating system (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″) or (CS5), wherein the oily phase of the emulsion comprises
40-60 wt-%, based on the total weight of the oily phase, of at least one enteric coating material, and
40-60 wt-%, based on the total weight of the oily phase, of at least one oil, which is solid at 20° C.

The present invention also relates to a coating system (CS6′″″), which is the coating system (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″) or (CS5), wherein the oily phase of the emulsion consists of
40-60 wt-%, based on the total weight of the oily phase, of at least one enteric coating material, and
40-60 wt-%, based on the total weight of the oily phase, of at least one oil, which is solid at 20° C., and
up to 15 wt-%, based on the total weight of the oily phase, of at least one further auxiliary agent.

The present invention also relates to a coating system (CS6″″″), which is the coating system (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″) or (CS5), wherein the oily phase of the emulsion consists of
40-60 wt-%, based on the total weight of the oily phase, of at least one enteric coating material, and
40-60 wt-%, based on the total weight of the oily phase, of at least one oil, which is solid at 20° C.

The present invention relates to a coating system (CS7), which is the coating system (CS6), (CS6′), (CS6′″) or (CS6″″″), wherein the auxiliary agent is chosen from the group consisting of colors, antifoaming agents, flavors and antioxidants.

As stated above all percentages always add up to 100% (this applies to all compositions disclosed herein).

In a preferred embodiment of the present invention, the gum having emulsifying properties is gum acacia (also known as gum Arabic

Therefore, the present invention also relates to a coating system (CS8), which is the coating system (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″), (CS5), (CS6), (CS6′), (CS6″), (CS6′″), (CS6″″), (CS6′″″), (CS6″″″) or (CS7), wherein the gum having emulsifying properties is gum acacia (also known as gum Arabic).

In a preferred embodiment of the present invention, the at least one enteric coating is Candelilla wax.

Therefore, the present invention also relates to a coating system (CS9), which is the coating system (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″), (CS5), (CS6), (CS6′), (CS6″), (CS6′″), (CS6″″), (CS6′″″), (CS6″″″), (CS7) or (CS8), wherein the at least one enteric coating is Candelilla wax.

In a preferred embodiment of the present invention, the at least one oil, which is solid at 20° C., is a hydrogenated oil and/or a fully hydrogenated oil.

In a more preferred embodiment of the present invention, the at the least one oil, which is solid at 20° C., is fully hydrogenated rapeseed oil (Canola oil).

Therefore, the present invention also relates to a coating system (CS10), which is the coating system (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″), (CS5), (CS6), (CS6′), (CS6″), (CS6′″), (CS6″″), (CS6′″″), (CS6″″″), (CS7), (CS8) or (CS9), wherein the at least one oil, which is solid at 20° C., is a hydrogenated oil and/or a fully hydrogenated oil.

Therefore, the present invention also relates to a coating system (CS10′), which is the coating system (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″), (CS5), (CS6), (CS6′), (CS6″), (CS6′″), (CS6″″), (CS6′″″), (CS6″″″), (CS7), (CS8) or (CS9), wherein at the least one oil, which is solid at 20° C., is fully hydrogenated rapeseed oil (Canola oil).

Candelilla wax is a wax derived from the leaves of the small Candelilla shrub native to northern Mexico and the southwestern United States, Euphorbia cerifera and Euphorbia antisyphilitica, from the family Euphorbiaceae. It is yellowish-brown, hard, brittle, aromatic, and opaque to translucent. Candelilla wax consists of mainly hydrocarbons (about 50%, chains with 29-33 carbons), esters of higher molecular weight (20-29%), free acids (7-9%), and resins (12-14%, mainly triterpenoid esters)

Rapeseed oil is a vegetable oil obtained from rapeseed. Rapeseed (Brassica napus subsp. napus), also known as rape, or oilseed rape, is a bright-yellow flowering member of the family Brassicaceae (mustard or cabbage family), cultivated mainly for its oil-rich seed, which naturally contains appreciable amounts of erucic acid. Canola is a group of rapeseed cultivars that were bred to have very low levels of erucic acid and are especially prized for human and animal food.

Hydrogenated rapeseed oil is obtained from native rapeseed oil by fat hydrogenation. Fat hydrogenation is the process of combining fat—typically vegetable oils—with hydrogen to make it more saturated. Their partial hydrogenation reduces most, but not all, of the carbon-carbon double bonds. The degree of hydrogenation is controlled by restricting the amount of hydrogen, reaction temperature and time, and the catalyst. Hydrogenation converts liquid vegetable oils into solid or semi-solid fats. Changing the degree of saturation of the fat changes some important physical properties, such as the melting range, which is why liquid oils become semi-solid.

Full hydrogenation results in a molecule containing the maximum amount of hydrogen (in other words, the conversion of an unsaturated fatty acid into a saturated one). Partial hydrogenation results in the addition of hydrogen atoms at some empty positions, with a corresponding reduction in the number of double bonds.

As stated above the particles are coated with the coating systems, which are the emulsions described above.

The emulsions used for the coating process should have a viscosity, which is suitable that the emulsion can be sprayed on the particles and that the spraying nozzle is not blocked.

A suitable range of viscosity goes up to 500 mPas. (measured at 45° C. (spraying temperature) using a Brookfield rheometer)

Therefore, the present invention relates to a coating system (CS11), which is the coating system (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″), (CS5), (CS6), (CS6′), (CS6″), (CS6′″), (CS6″″), (CS6′″″), (CS6″″″), (CS7), (CS8), (CS9) (CS10) or (CS10′), wherein the emulsion has a viscosity of up to 500 mPas.

The coating process can be done by using commonly known processes and equipment. Also, the process conditions applied are commonly known and vary depending which process and/or equipment is used.

Typical coating techniques for coating of such tablets are i.e. dip coating, spray coating, film coating fluid bed coating, drum coating and pan coating

It is not essential for the present invention, which process is used.

Preferably, spray coating, drum coating or film coating is used.

Any suitable coating device can be used.

Such equipment is commercially available from a variety of suppliers, such i.e. Glatt (CH), Comasa etc.

The coating process is usually carried out as following
(i) the emulsion (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″), (CS5), (CS6), (CS6′), (CS6″), (CS6′″), (CS6″″), (CS6′″″), (CS6″″″), (CS7), (CS8), (CS9) (CS10), (CS10′) or (CS11) is prepared by preparing each of the phases, which are then emulsified, then
(ii) the emulsion is sprayed onto the particles (at a temperature range of 20° C. to 70° C.), then
(iii) the coated particles are collected and then
(iv) optionally the coated particles are undergoing a curing process.

As stated above, the process parameters chosen for the coating are commonly known and used for such a process.

The curing process is usually done by heating the particles up to 90° C. for a period of at least 1 minute. The maximal duration of the curing step is not essential, but it is usually not more than one hour.

Therefore, the curing step is carried for 1 minute to up to one hour.

Preferably the coated particles are undergoing a curing process step.

The coating process is usually carried out as follows:
(i) the emulsion (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″), (CS5), (CS6), (CS6′), (CS6″), (CS6′″), (CS6″″), (CS6′″″), (CS6″″″), (CS7), (CS8), (CS9) (CS10), (CS10′) or (CS11) is prepared by preparing each of the phases, which are then emulsified, then
(ii) the emulsion is sprayed onto the particles (at a temperature range of 20° C. to 70° C.), then
(iii) the coated particles are collected and then
(iv) the coated particles are undergoing a curing process at a temperature of up to 90° C. for at least one minute (preferably 1 minute to up to one hour).

When the coating system is applied onto the particle, the water of the emulsion is (more or less) evaporated. This means that the water content of the coating layer of coated particle is less than 5 wt-%, based on the total weight of the coated particle (preferably below 3 wt-%, more preferred below 2 wt-%, even more preferred below 1 wt-%).

The coated particles according to the present invention consists of
(a) a core (which is the particle) and
(b) the coating layer, which is the coating system (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″), (CS5), (CS6), (CS6′), (CS6″), (CS6′″), (CS6″″), (CS6′″″), (CS6″″″), (CS7), (CS8), (CS9) (CS10), (CS10′) or (CS11),
   wherein the coated particles comprise less than 5 wt-% of water, based on the total weight of the coated particles (preferably less than 3 wt-%, more preferred less than 2 wt-%, even more preferred less than 1 wt-%, based on the total weight of the coated particles).

The coated particle according to the present invention consists of
(a) 60 to 99 wt-%, based on the total weight of the coated particle, of the particle comprising at least one active ingredient, and
(b) 1 to 40 wt-% (20-35), based on the total weight of the coated particle, of the coating layer, which is the coating system (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″), (CS5), (CS6), (CS6′), (CS6″), (CS6′″), (CS6″″), (CS6′″″), (CS6″″″), (CS7), (CS8), (CS9) (CS10), (CS10′) or (CS11),
   wherein the coated particles comprise less than 5 wt-% of water, based on the total weight of the coated particles (preferably less than 3 wt-%, more preferred less than 2 wt-%, even more preferred less than 1 wt-%, based on the total weight of the coated particles).

Therefore, the present invention relates to a coated particle (CP) consisting of
(a) 60 to 99 wt-%, based on the total weight of the coated particle, of the particle comprising at least one active ingredient, and
(b) 1 to 40 wt-%, based on the total weight of the coated particle, of the coating layer, which is the coating system (CS), (CS′), (CS″), (CS′″), (CS1), (CS1′), (CS1″), (CS1′″), (CS2), (CS2′), (CS3), (CS3′), (CS4), (CS4′), (CS4″), (CS4′″), (CS4″″), (CS5), (CS6), (CS6′), (CS6″), (CS6′″), (CS6″″), (CS6′″″), (CS6″″″), (CS7), (CS8), (CS9) (CS10), (CS10′) or (CS11),
   wherein the coated particles comprise less than 5 wt-% of water, based on the total weight of the coated particles (preferably less than 3 wt-%, more preferred less than 2 wt-%, even more preferred less than 1 wt-%, based on the total weight of the coated particles).

The particles, which are coated by the inventive coating system, may have any size and shape, which are meant to be consumed.

As stated above, the shape of the particle (which is the core of the coated particle) does not change substantially when it is coated.

The particles may have a size, which is commonly known for the type of particles. They may be in the form of a monolithic dosage form or multiparticulates (or multiple unit dosage form).

Therefore, the present invention relates to coating particles (CP1), which are the coated particles (CP), wherein the coated particles are a monolithic dosage form or multiparticulates (or multiple unit dosage form).

The size of the largest dimension relates on the shape of the particle.

The shape can be any known and used, such as spheres, disc-like, bean-like, egg shape etc.

A suitable size range goes from below 1 mm to about 1.5 cm (largest dimension).

Therefore, the present invention relates to a coating particle (CP2), which is the coated particle (CP) or (CP1), wherein the coated particle has a size from below 1 mm to about 1.5 cm (largest dimension).

The particle, which is coated, can be a tablet, capsule, caplet, extrudate, pellet, globuli, granulate, beadlet, etc which comprises at least one active ingredient and optionally at least one further excipient, which are used to produce such a dosage form.

Therefore, the present invention relates to a coating particle (CP3), which is the coated particle (CP), (CP1) or (CP2), wherein the particle, which is coated, is a tablet, capsule, caplet, extrudate, pellet, globuli, granulate, or a beadlet, which comprises at least one active ingredient and optionally at least one further excipient, which are used to produce such a dosage form.

The particle can also be a pure substance. This means no excipient is used.

The coating layer covers the particle (more or less) equally. This means that the particle is coated completely, and the thickness is more or less the same all over the particle.

The thickness of the coating layer is from 20 μm to 500 μm (preferably 50 μm to 500 μm, more preferably 100 μm to 400 μm).

The thickness is measured by commonly known methods, such as i.e., scanning electron microscopy.

Therefore, the present invention relates to a coating particle (CP4), which is the coated particle (CP), (CP1), (CP2) or (CP3), wherein the thickness of the coating is 20 μm to 500 μm (preferably 50 μm to 500 μm, more preferably 100 μm to 400 μm).

The active ingredient in the particle (core) can be any suitable one depending on the desired use of the coated particle.

The active ingredient can be a i.e., pharmaceutical compound (a drug), a vitamin, a mineral, probiotic, nutritional lipid, enzyme, eubiotic, plant extract and nutraceutical.

Therefore, the present invention relates to a coating particle (CP5), which is the coated particle (CP), (CP1), (CP2), (CP3) or (CP4), wherein the active ingredient in the particle is chosen from the group consisting of pharmaceutical compound (a drug), a vitamin, a mineral, probiotic, nutritional lipid, enzyme, eubiotic, plant extract and nutraceutical.

When a vitamin is present, the vitamin can be a water-soluble and/or a fat-soluble vitamin.

Water soluble vitamins are ascorbic acid (vitamin C), thiamin, riboflavin, niacin, vitamin B6 (pyridoxine, pyridoxal, and pyridoxamine), folacin, vitamin B12, biotin, and pantothenic acid.

Fat soluble vitamins are A, vitamin D, vitamin E and vitamin K.

The amount of the at least one active ingredient in the particle can vary depending on the active ingredient and/or on the use of the active ingredient and/or on the dosage regime of the final formulation (which is consumed), etc.

Depending on the active ingredient in the particle (which is the core of the coated particle), the coated particle can be used in various fields of application, such as in a dietary supplement formulation, in a pharmaceutical formulation, in a personal care formulation, in food formulation and feed formulation.

Therefore, the present invention relates to the use of at least one coated particle, which is the coated particle (CP), (CP1), (CP2), (CP3), (CP4) or (CP5) in a dietary supplement formulation, in a pharmaceutical formulation, in a personal care formulation, in food formulation and/or feed formulation.

The coated particle according to the present invention can be used as such, or it can be incorporated into a formulation, which can be any commonly known formulation (preferably, it is a dry formulation).

The following examples serve to illustrate specific embodiments of the invention claimed herein. All percentages are given in relation to the weight, and all the temperatures are given in degrees Celsius.

### EXAMPLES

### Example 1

### Aqueous Phase Preparation:

The Acacia gum solution (5%) was prepared beforehand by progressively dispersing 40 g of Acacia gum spray-dried powder in 800 g of deionised water at 45° C. The solution was left under stirring at 700 rpm for 1 h.

250 g of this solution was used, which has been sieved with a sieve (mesh size around 500 μm) before being transferred in a 600 mL glass beaker. The beaker was finally immersed in a water bath at 90° C. to raise the aqueous phase temperature.

### Oily Phase Preparation:

Two metallic beakers, one containing the wax and the other the Rapeseed oil, were placed in an oven 2 h before the preparation to melt both products. Once melted, 55.15 g of Candelilla wax and 55.15 g of Rapeseed oil have been weighted in the same 250 mL glass beaker. Then, the mixture has been quickly placed in a water bath at 90° C. under magnetic stirring to mix both products.

### Emulsion Preparation:

Once the temperature of the aqueous phase was greater than 65° C., the homogenizer speed (Polytron PT6000) was progressively increased until 21700 rpm while adding the prepared oily phase slowly. The emulsion was mixed for 30 min.

The emulsion was finally sieved using a sieve (mesh size around 500 μm) and transferred in a 250 mL Duran Schott bottle, beforehand tared and pre-heated in the oven at 90° C.

### Coating:

The coating has been performed in a drum coater Glatt GC 1 equipped with a 0.8 L drum and the standard 0.8 mm nozzle. A Silicone tubing (2×2×6) from VWR was used to ensure the transportation of the emulsion from the bottle to the nozzle. A water bath was used to keep the emulsion hot during the coating. The drum was filled with 250 g of Vitamin B2 tablets, which were used as core particle.

### Post Coating Curing:

The tablets were collected after the coating and were cured in an oven (Memmert UNB 200) at 85° C. for 30 min.

The following table shows the composition of the coating system used in Example 1

| Phase | Ingredients | Amount (% w/w) |
|---|---|---|
| Aqueous | Water | 65.9 |
| | Acacia gum | 3.5 |
| Oily | Candelilla wax | 15.3 |
| | Rapeseed oil (fully hydro- | 15.3 |
| | genated) | |

The coated particles have been tested to demonstrate the controlled release property

Release test performed with USP apparatus 3 equipped with vessels containing buffer solutions at 37.5° C. without enzymes

| | | Time | Dip per | | Medium: | Medium: Volume/ |
|---|---|---|---|---|---|---|
| | Simulation | [hh:mm] | minute | Medium: pH | Enzymes | Vessel [ml] |
| Row1 | wait Position | — | — | — | — | — |
| Row2 | Stomach | 2:00 | 30 | 3.2 | n/a | 230 |
| Row3 | Duodenum | 3:00 | 15 | 6.8 | n/a | 230 |
| Row4 | jejunum/ | 0:30 | 15 | 7.3 | n/a | 230 |
| | ileum | | | | | |

The results of this test can be found in FIG. 1

Release tests performed with an USP apparatus 3 equipped with vessels containing buffer solutions at 37.5° C. with enzymes:

| | | Time | Dip per | Medium: | | Medium: Volume/ |
|---|---|---|---|---|---|---|
| | Simulation | [hh:mm] | minute | pH | Medium: Enzymes | Vessel [ml] |
| Row1 | wait Position | — | — | — | — | — |
| Row2 | Stomach | 2:30 | 30 | 3.2 | Pepsin (from Amresco): | 230 |
| | | | | | 157 mg/230 ml | |
| Row3 | Duodenum | 3:00 | 15 | 6.8 | Pancreatine (from Sigma | 230 |
| | | | | | Aldrich): 422 mg/230 ml | |
| | | | | | bile salts: 948 mg/230 ml | |
| Row4 | jejunum/ | 0:30 | 15 | 7.3 | Pancreatine (from Sigma | 230 |
| | ileum | | | | Aldrich): 422 mg/230 ml | |
| | | | | | bile salts: 948 mg/230 ml | |
| Row5 | Proximal | 8:00 | 15 | 6.4 | Viscozyme (from Sigma | 230 |
| | colon | | | | Aldrich): 1.75 ml/230 ml | |

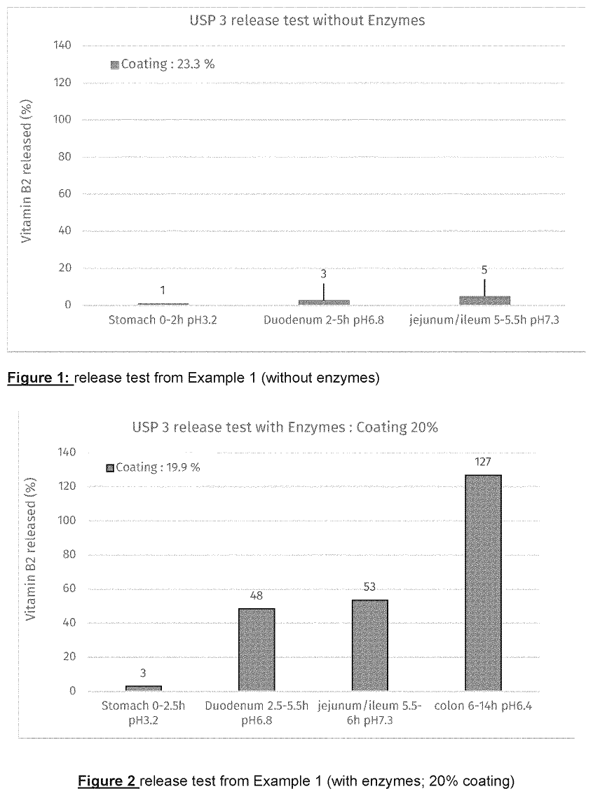

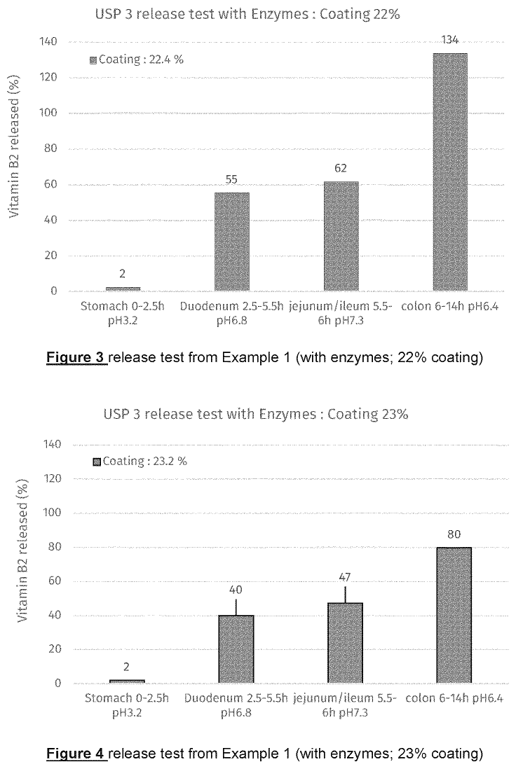

The results of these tests (for 20%, 22% and 23% of coating) can be found in FIGS. 2, 3, and 4.

### Example 2: Composition without Fat (Comparison Example)

### Aqueous Phase Preparation:

The Acacia gum solution (5%) was prepared beforehand by progressively dispersing 40 g of Acacia gum spray-dried powder in 800 g of deionised water at 45° C.

The solution was left under stirring at 700 rpm for 1 h before.

200 g of this solution was used, which has been sieved with a sieve (mesh size around 500 μm) before being transferred in a 400 mL glass beaker. The beaker was finally immersed in a water bath at 90° C. to raise the aqueous phase temperature.

### Oily Phase Preparation:

5.88 g of Glycerol were firstly weighed in a 150 mL glass beaker. Then, 88.24 g of melted Candelilla wax were added to the beaker, which was quickly placed in a water bath at 90° C. under stirring to keep the wax liquid and mix both products.

### Emulsion Preparation:

Once the temperature of the aqueous phase was greater than 65° C., the homogenizer speed (Ultraturrax T25) was progressively increased until 24000 rpm while adding the oily phase slowly. The emulsion was mixed for 30 min.

The emulsion was finally sieved using a sieve (mesh size 500 μm) and transferred in a 250 mL Duran Schott bottle, beforehand tared and pre-heated in the oven at 90° C.

### Coating:

The coating has been performed in a drum coater Glatt GC 1 equipped with a 0.8 L drum and the standard 0.8 mm nozzle. A Silicone tubing (2×2×6) from VWR was used to ensure the transportation of the emulsion from the bottle to the nozzle. A water bath was used to keep the emulsion hot during the coating. The drum was filled with 300 g of Vitamin B2 tablets.

### Post Coating Curing:

Few tablets collected after the coating were cured in an oven (Memmert UNB 200) at 75° C. for the night, but also at 85° C. for 30 min, to reduce the microporosity within the coating.

The following table shows the composition of the coating system used in Example 2 (comparison Example)

| Phase | Ingredients | Amount (% w/w) |
|---|---|---|
| Aqueous | Water | 64.6 |
| | Acacia gum | 3.4 |
| Oily | Candelilla wax | 30 |
| | Glycerol | 2 |

Release test performed with USP apparatus 3 equipped with vessels containing buffer solutions at 37.5° C. without enzymes:

| | | Time | Dip per | | Medium: | Medium: Volume/ |
|---|---|---|---|---|---|---|
| | Simulation | [hh:mm] | minute | Medium: pH | Enzymes | Vessel [ml] |
| Row1 | wait Position | — | — | — | — | — |
| Row2 | Stomach | 2:00 | 30 | 3.3 | n/a | 230 |
| Row3 | Duodenum | 3:00 | 15 | 6.8 | n/a | 230 |
| Row4 | jejunum/ | 0:30 | 15 | 7.3 | n/a | 230 |
| | ileum | | | | | |
| Row5 | Proximal | 8:00 | 15 | 6.5 | n/a | 230 |
| | colon | | | | | |

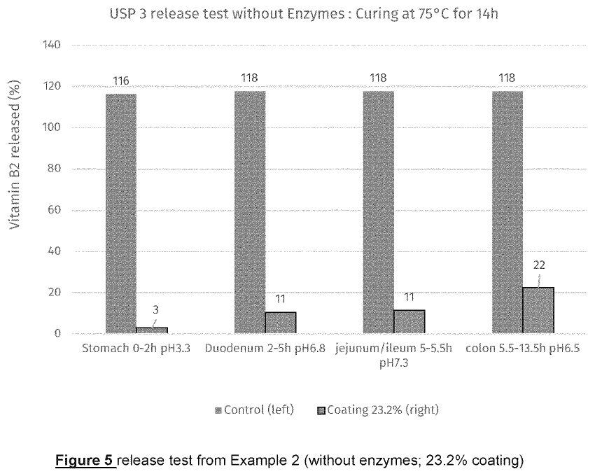

The results of this test can be found in FIG. 5

Release test performed with USP 3 apparatus equipped with vessels containing buffer solutions at 37.5° C. with enzymes

| | | Time | Dip per | Medium: | | Medium: Volume/ |
|---|---|---|---|---|---|---|
| | Simulation | [hh:mm] | minute | pH | Medium: Enzymes | Vessel [ml] |
| Row1 | wait Position | — | — | — | — | — |
| Row2 | Stomach | 2:30 | 30 | 3.2 | Pepsin (from Amresco): | 230 |
| | | | | | 157 mg/230 ml | |
| Row3 | Duodenum | 3:00 | 15 | 6.8 | Pancreatine (from Sigma | 230 |
| | | | | | Aldrich): 422 mg/230 ml | |
| Row4 | jejunum/ | 0:30 | 15 | 7.3 | Pancreatine (from Sigma | 230 |
| | ileum | | | | Aldrich): 422 mg/230 ml | |
| Row5 | Proximal | 8:00 | 15 | 6.5 | Viscozyme (from Sigma | 230 |
| | colon | | | | Aldrich): 1.75 ml/230 ml | |

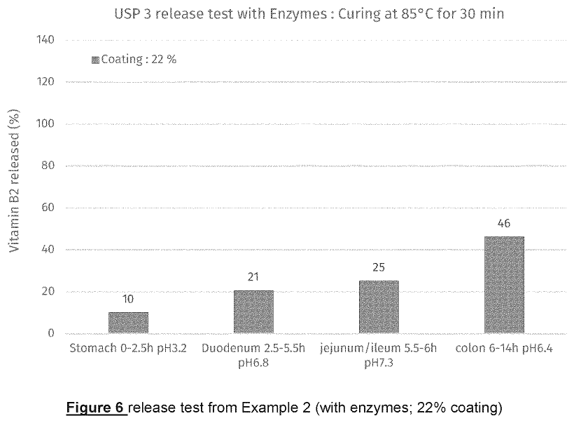

The results of this test can be found in FIG. 6.

It can be seen that the Examples of the present invention show a much better release properties than the comparison Examples

1. A coating system comprising an
(i) aqueous solution comprising
a. water, and
b. at the least one gum having emulsifying properties, and an
(ii) oily phase comprising
a. at least one wax, and
b. at least one oil, which is solid at 20° C.

2. The coating system according to claim 1, wherein the emulsion consists of 50-80 wt-%, based on the total weight of the emulsion, of the aqueous (water) phase and 20-50 wt-%, based on the total weight of the emulsion, of the oily phase.

3. The coating system according to claim 1, wherein the aqueous phase of the emulsion comprises
65-90 wt-%, based on the total weight of the aqueous phase, of water and
0.5-10 wt-%, based on the total weight of the aqueous phase, of at the least one gum having emulsifying properties,
and
up to 15 wt-%, based on the total weight of the aqueous phase, of at least one further auxiliary agent.

4. The coating system according to claim 1, wherein the aqueous phase of the emulsion consists of
75-99 wt-%, based on the total weight of the aqueous phase, of water and
1-25 wt-%, based on the total weight of the aqueous phase, of at the least one gum having emulsifying properties, and
up to 15 wt-%, based on the total weight of the aqueous phase, of at least one further auxiliary agent.

5. The coating system according to claim 1, wherein the oily phase of the emulsion comprises
30-70 wt-%, based on the total weight of the oily phase, of at least one enteric coating material, and
30-70 wt-%, based on the total weight of the oily phase, of at least one oil, which is solid at 20° C., and
up to 15 wt-%, based on the total weight of the oily phase, of at least one further auxiliary agent.

6. The coating system according to claim 1, wherein the oily phase of the emulsion consists of
30-70 wt-%, based on the total weight of the oily phase, of at least one enteric coating material, and
30-70 wt-%, based on the total weight of the oily phase, of at least one oil, which is solid at 20° C., and
up to 15 wt-%, based on the total weight of the oily phase, of at least one further auxiliary agent.

7. The coating system according to claim 1, wherein the at least one gum having emulsifying properties is gum accacia.

8. The coating system according to claim 1, wherein the at least one enteric coating is a wax.

9. The coating system according to claim 1, wherein the at least one oil, which is solid at 20° C., is full hydrogenated rapeseed oil (Canola oil).

10. The coating system according to claim 1, wherein the emulsion has a viscosity of up to 500 mPas.

11. Coating process wherein
(i) an emulsion of claim 1 is prepared by preparing each of the phases, which are then emulsified, then
(ii) the emulsion is sprayed onto the particles (at a temperature range of 20° C. to 70° C.), then
(iii) the particles are collected and then
(iv) optionally the particles are undergoing a curing process.

12. A coated particle consisting of
(a) 70 to 99 wt-%, based on the total weight of the coated particle, of the particle comprising at least one active ingredient
(b) 1 to 30 wt-%, based on the total weight of the coated particle, of the coating layer, which is the coating system according to claim 1,
wherein the coated particle comprises less than 5 wt-% of water, based on the total weight of the coated particle.
